(19) United States
(12) Patent Application Publication
(10) Pub. No.: US 2011/0081686 A1
(43) Pub. Date: Apr. 7, 2011

(54) Reactor for Bulk Manufacture of PCR Amplicons

(75) Inventors: C. Marston MANTHORPE, San Diego, CA (US); Mark E. MINICK, San Diego, CA (US); Edward M. DOMANICO, San Diego, CA (US)

(73) Assignee: Vical Incorporated, San Diego, CA (US)

(21) Appl. No.: 12/895,441

(22) Filed: Sep. 30, 2010

Related U.S. Application Data
(60) Provisional application No. 61/247,887, filed on Oct. 1, 2009.

(51) Int. Cl. C12P 19/34; C12M 1/38; C12M 1/00

(52) U.S. Cl. 435/91.2; 435/286.1; 435/286.5; 435/289.1

(57) ABSTRACT

A reactor and methods of use are disclosed for amplification of an amplicon. The reactor includes a housing having a fluid path with a fluid inlet and a fluid outlet and one or more reaction chambers suitable for housing the amplicon. The reaction chambers are positioned within the housing in thermal communication with a fluid flowing in the fluid path to heat and/or cool the reaction chambers and the inside of the reaction chambers are coated with or are a plastic material. A variable temperature fluid source is in fluid communication with the fluid inlet and the fluid source is configured to provide fluid at temperatures suitable for amplification, including a denature temperature, an anneal temperature, and an extension temperature. A controller is coupled to the variable temperature fluid source to control a fluid temperature within the fluid path by controlling the variable temperature fluid source.

### CROSS-REFERENCES TO RELATED APPLICATIONS

This application claims priority under 35 U.S.C. §119(e) to U.S. Provisional Application No. 61/247,887, filed Oct. 1, 2009, which is related to U.S. patent application Ser. No. 11/949,745, entitled “Linear Expression Cassette Vaccines”, filed Dec. 3, 2007, both of which are fully incorporated herein by reference.

### GRANT INFORMATION

Some work described herein was partially funded by DARPA grant W911NF-05-1-0545. The U.S. Federal Government has certain rights in the disclosed invention.

### FIELD OF THE DISCLOSURE

The present invention relates to a reactor for the bulk manufacture of PCR amplicons, as well as methods of amplifying PCR amplicons.

### BACKGROUND OF THE INVENTION

DNA vaccines have proven effective in a growing number of infectious disease indications. For example, influenza is a highly contagious illness of the respiratory tract caused by RNA viruses of the Orthomyxoviridae family (Knipe & Howley). According to the Centers for Disease Control and Prevention, five to 20% of the U.S. population gets influenza every year, with about 36,000 deaths annually due to complications from the infection. Influenza is among the top seven leading causes of death in the U.S. despite over 60 years of licensed influenza vaccine availability; still, the most effective protection against influenza infection is vaccination. Small periodic changes in the virus surface antigens (antigenic drift) require the development of new vaccines every season. Moreover, major changes in the virus surface proteins (antigenic shift) can result in highly virulent strains that have the potential to cause a pandemic. Outbreaks of influenza in animals increase the chances of a pandemic, through the reshuffling of animal and human influenza virus genomes resulting in a new virus strain. A recent pandemic outbreak of a new strain of an influenza A virus subtype H1N1, officially referred to as the novel H1N1, first identified in April 2009 has raised new concerns of current vaccination strategies. It has become clear that successful containment of an outbreak of a highly virulent influenza strain will require fast manufacture of large quantities of vaccines.

While vaccination against influenza has been reported as the most cost-effective approach, development and manufacture of influenza vaccines require the use of technologies that have proven slow and unreliable and are therefore inadequate to meet the challenges of a potentially rapidly changing and spreading pandemic. One potential technology that may meet the requirements for rapid manufacture of vaccines is plasmid DNA (pDNA). However, pharmaceutical grade pDNA production requires bacterial fermentation followed by lengthy purification and extensive quality control testing. There are also some concerns that trace amounts of antibiotics and other fermentation components may carry over after purification. Ideally, a nucleic acid vaccine could be produced using a cell-free system more akin to a small molecule synthetic process.

There is a need for an amplicon reactor to automate the amplification process and quickly amplify an amplicon such as but not limited to a piece of linear RNA or alternatively DNA that may or may not encode a gene product.

### BRIEF SUMMARY OF THE INVENTION

The present invention is directed to a reactor used for amplification of a piece of DNA or RNA, also generally referred to herein as an amplicon. Two types of reactors are disclosed, a batch amplicon reactor in which the amplicon being part of a PCR mixture is heated and cooled in a reaction chamber, and a continuous flow reactor in which the amplicon being part of a PCR mixture is heated and cooled as it flows through heat exchanger modules having different temperatures.

In one embodiment, a reactor is disclosed for the production of a linear expression cassette (LEC), that is, a linear piece of DNA that is capable of expressing a gene product, from a polymerase chain reaction (PCR) mix. The reactor includes a housing having at least one reaction chamber therein surrounded by a fluid path for heat transfer. The heat transfer fluid necessary for heat transfer enters the housing through a fluid inlet and exists by way of a fluid outlet. The reaction chambers, storing the PCR reaction mixture, are positioned within the housing in thermal communication with the heat transfer fluid (HT fluid) flowing in the fluid path to heat and/or cool the reaction chambers. A variable temperature HT fluid source is in fluid communication with the fluid inlet and the HT fluid source is configured to provide HT fluid at temperatures suitable for amplification, including a denature temperature, an anneal temperature, and/or an extension temperature. A controller is coupled to the variable temperature HT fluid source to control a HT fluid temperature within the fluid path by controlling the variable temperature HT fluid source.

In some embodiments, the variable temperature HT fluid source is a plurality of HT fluid baths at different temperatures with outlet valves in fluid communication with the fluid inlet of the housing. The controller opens and closes the outlet valves of the HT fluid baths to control the fluid temperature within the housing.

In some embodiments, the variable temperature HT fluid source is a steam source and a liquid source coupled to a steam/water mixing valve in fluid communication with the fluid inlet. The controller adjusts the steam/water mixing valve to control the HT fluid temperature within the housing.

In another embodiment, a method of amplification of an amplicon to produce a linear expression cassette (LEC) is disclosed using a batch reactor. The method includes providing a reactor having a housing, one or more reaction chambers positioned within the housing in thermal communication with the HT fluid flowing in a fluid path to heat and/or cool the reaction chambers, a variable temperature HT fluid source to provide HT fluid at temperatures suitable for amplification, including a denature temperature, an anneal temperature, and/or an extension temperature, and a controller coupled to the variable temperature HT fluid source to control the HT fluid temperature within the fluid path by controlling the variable temperature HT fluid source. The PCR reaction mixture including the amplicon is placed within the one or more reaction chambers and then heated and cooled within the reaction chambers for an amplification process cycle by flowing HT fluid in the fluid path at a plurality of temperatures and time periods in order to produce a linear expression cassette.

In another embodiment, a continuous flow reactor is disclosed for amplification of an amplicon. The reactor includes a plurality of heat exchanger modules constructed of plate heat exchangers, with each heat exchanger module having an amplicon fluid path combinable into a continuous amplicon fluid path passing through the plurality of heat exchanger modules. A plurality of HT fluid sources provide heating/cooling fluids to a heating/cooling fluid path of each heat exchanger module at temperatures suitable for amplification, including a denature temperature, an anneal temperature, and/or an extension temperature. A controller coupled to the plurality of HT fluid sources is configured to control a temperature within each of the plurality of heat exchanger modules by controlling an HT fluid temperature and/or fluid flow rate through the heating/cooling fluid path.

### BRIEF DESCRIPTION OF THE DRAWINGS

FIG. 1 is a schematic representation of a reactor for bulk manufacture of PCR amplicons in which the reactor housing utilizes water baths for heating/cooling the amplicon.

FIG. 2 is a schematic representation of an alternate embodiment of a reactor for bulk manufacture of PCR amplicons in which the reactor housing uses a steam/water mixing valve coupled to a steam source and a liquid source for heating/cooling the amplicon.

FIG. 3 is a schematic representation of an alternate embodiment of a reactor for bulk manufacture of PCR amplicons using multiple reactor housings.

FIG. 4 illustrates a cross-sectional view taken along line 4-4 of FIG. 2 showing a first embodiment of a reactor housing comprising a shell having a bundle of reaction chambers positioned within it.

FIG. 5 illustrates a cross-sectional view taken along line 4-4 of FIG. 2 showing an alternate embodiment of a reactor housing comprising a shell having a perforated cage for maintaining and positioning reaction chambers.

FIG. 6 is a top view of a perforated plate used in construction of the cage illustrated in FIG. 5.

FIG. 7 illustrates a cross-sectional view taken along line 4-4 of FIG. 2 showing an alternate embodiment of a reactor housing comprising a shell having a perforated cage for maintaining and positioning reaction chambers.

FIG. 8 is a schematic representation of an alternate embodiment of a reactor housing comprising a shell having a bundle of tubes positioned within. A first fluid runs through the tubes, and a second fluid flows over the tubes (through the shell) to transfer heat between the two fluids.

FIGS. 9A, 9B and 9C is a schematic representation of a continuous flow reactor for bulk manufacture of PCR amplicons wherein amplicons pass through heat exchanger modules at different temperatures, heating and/or cooling the amplicon for amplification.

FIG. 10 is a schematic representation of an alternate embodiment of a continuous flow reactor for bulk manufacture of PCR amplicons wherein amplicons pass through heat exchanger modules at different temperatures in which a PCR reaction mixture including the amplicon enters an inlet, flows through multiple heat exchanger modules in a continuous fluid path and exits an outlet as amplified LEC.

### DETAILED DESCRIPTION OF THE INVENTION

The present invention is directed to a reactor for the bulk manufacture of PCR amplicons. The amplicon to be amplified may be a linear piece of DNA or RNA and may be capable of expressing a polypeptide. The amplicon is heated and cooled for amplification. An amplification cycle of the amplicon may include the following: a denature step, an annealing step, and an extension step. The PCR reaction carried out according to the present invention is thus capable of producing bulk quantities of the desired amplicon or template. Thus, depending on the amplicon it is possible to generate commercial quantities of that particular amplicon. Large quantities of an amplicon would be useful in a wide variety of applications, such as but not limited to, linear expression cassettes that may be useful as vaccines; oligonucleotides that may be useful as adjuvants by themselves or with vaccines, inhibitory or stimulatory DNA pharmaceuticals, and linear expression cassette transfections.

Two types of reactors are disclosed herein: a batch reactor and a continuous flow reactor. At the heart of the batch reactor disclosed in the present invention there exists a heat exchanger of which there can be many variations on the shell and tube design. A shell and tube heat exchanger is a class of heat exchanger designs and as its name implies, this type of heat exchanger consists of a shell (a large pressure vessel) with a bundle of tubes inside it. One fluid runs through the tubes, and another fluid flows over the tubes (through the shell) to transfer heat between the two fluids. The set of tubes is called a tube bundle, and may be composed by several types of tubes: plain, longitudinally finned, etc. There can be many variations on the shell and tube design. Typically, the ends of each tube are connected to plenums (sometimes called water boxes) through holes in tube sheets. The tubes may be straight or bent in the shape of a U, called U-tubes. Most shell-and-tube heat exchangers are either 1, 2, or 4 pass designs on the tube side. This refers to the number of times the fluid in the tubes passes through the fluid in the shell. In a single pass heat exchanger, the fluid goes in one end of each tube and out the other.

Alternatively, the tubes may be closed at both ends thereby forming a plurality of individual reaction chambers. In this particular embodiment the PCR mixture containing the desired amplicon is placed within reaction chambers and a thermal heat transfer fluid (HT fluid) flows around the chambers, heating and cooling the amplicon for amplification. In the continuous flow reactor, the PCR mixture containing the desired amplicon flows through heat exchanger modules at different temperatures, heating and/or cooling the amplicon for amplification.

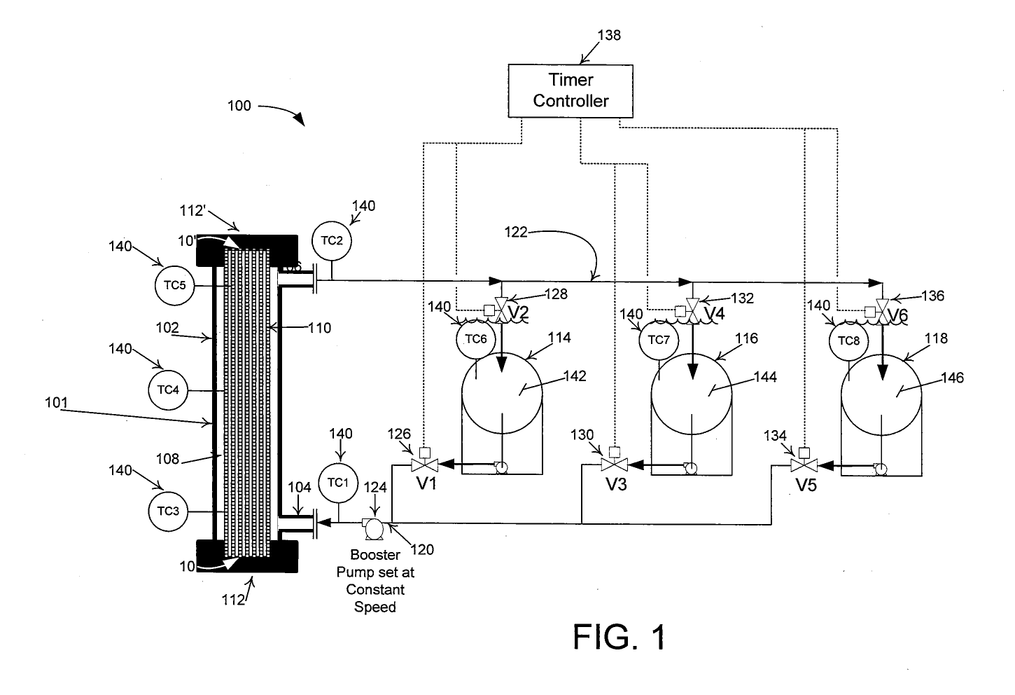

FIG. 1 shows one embodiment of a batch reactor 100 according to the present invention. The reactor 100 includes a heat exchange unit 101 comprising a reactor housing or shell 102 having a fluid inlet 104 and a fluid outlet 106 with a fluid path 108 there between. A set of tubes, referred to herein as a tube bundle, are positioned within reactor housing 102. Each tube has two ends 10 and 10′ and when these ends 10 and 10′ are sealed a reaction chamber 110 is formed. In one example, each tube end 10 comprising a tube bundle, may be sealed thus allowing the PCR mixture containing the desired amplicon to be loaded into the tubes prior to positioning the tube bundle within reactor housing 102. In this instance, tube ends 10′ could then be sealed after the PCR reaction mixture is loaded into the tube but prior to the tube being positioned with the reactor housing 102. Alternatively, the tube bundle could be positioned with the reactor housing 102 and then tube end 10′ could be sealed by end cap 112′. In another embodiment, a tube bundle is positioned within reactor housing 102 and tube end 10 is sealed by virtue of being in contact with end cap 112. The PCR reaction mixture is then loaded into the tube through tube end 10′ and then reaction chamber 110 is formed by sealing tube end 10 by positioning end cap 112′ over the top of the tube bundle. Reaction chambers 110 within reactor housing 102 are thus positioned within fluid path 108 such that the HT fluid flows around reaction chambers 110 thereby transferring heat from the HT fluid to the PCR reaction mixture confined within reaction chambers 110 through the reaction chamber side walls. There are several thermal design features that are to be taken into account when a heat exchange unit comprising a series of reaction chambers 110 positioned within housing 102 is utilized in a reactor for bulk manufacture of PCR amplicons. These include:
Tube diameter: Using a small tube diameter makes the heat exchanger both economical and compact. However, it is more likely for the heat exchanger to foul up faster and the small size makes mechanical cleaning of the fouling difficult. To prevail over the fouling and cleaning problems, larger tube diameters can be used. Thus to determine the tube diameter, the available space, cost and the fouling nature of the fluids must be considered. Tube diameter will also impact the volume of the PCR reaction mixture being loaded into the tube and this too will have an economical impact.
Tube thickness: The thickness of the wall of the tubes is usually determined to ensure: (i) axial strength; (ii) hoop strength (to withstand internal tube pressure); and (iii) buckling strength (to withstand overpressure in the shell).
Tube length: heat exchangers are usually cheaper when they have a smaller shell diameter and a long tube length. Thus, typically there is an aim to make the heat exchanger as long as physically possible whilst not exceeding production capabilities. However, there are many limitations for this, including the space available at the site where it is going to be used and the need to ensure that there are tubes available in lengths that are twice the required length (so that the tubes can be withdrawn and replaced). Also, it has to be remembered that long, thin tubes are difficult to take out and replace.
Tube corrugation: this type of tubes, mainly used for the inner tubes, increases the turbulence of the fluids and the effect is very important in the heat transfer giving a better performance.
Tube Layout: refers to how tubes are positioned within the shell. There are four main types of tube layout, which are, triangular)(30°, rotated triangular)(60°, square)(90° and rotated square)(45°. The triangular patterns are employed to give greater heat transfer as they force the fluid to flow in a more turbulent fashion around the piping. Square patterns are employed where high fouling is experienced and cleaning is more regular.

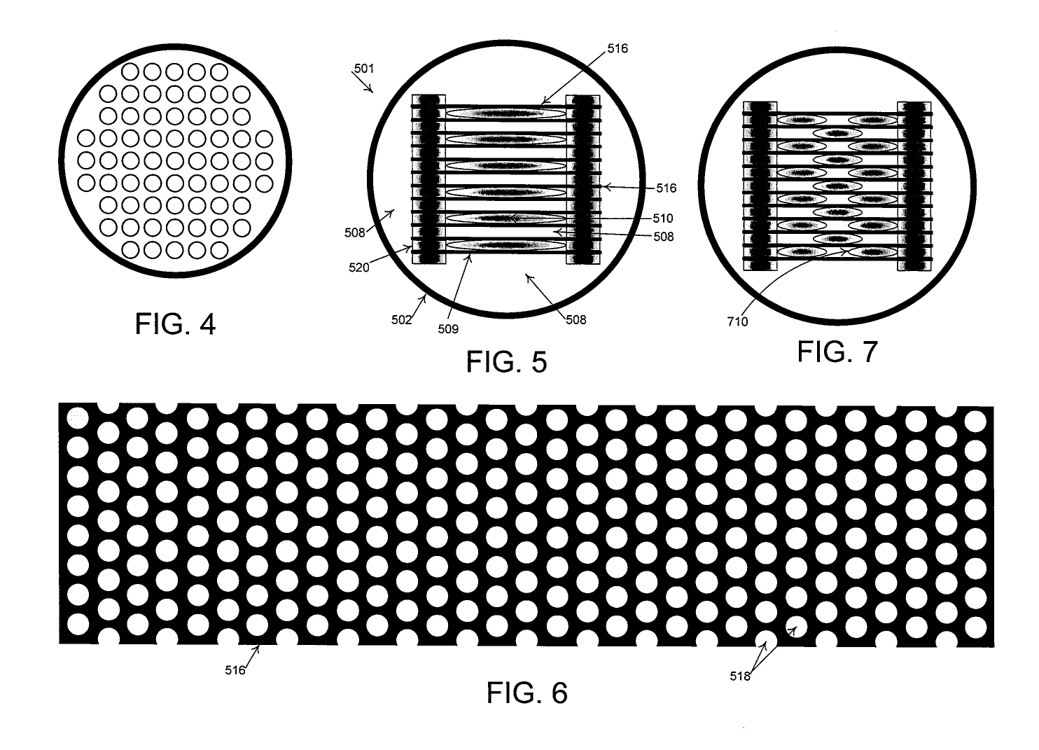

Taking into account the thermal design features discussed above reactor housing 102 and reaction chambers 110 may be any suitable size and shape and be made of any material having a high ratio of thermal conductivity. Typically, the tubes which make up reaction chambers 110 are double or single tube sheet, cylindrical in shape and when bundled together and positioned within reaction chamber 110 are best shown in FIG. 4. Reaction chambers 110 may be constructed of materials having a high ratio of thermal conductivity, such as but not limited to titanium, nickel, stainless steel, or copper. Reaction chambers 110 may also be constructed of thin materials having a low ratio of thermal conductivity, such as but not limited to PFA (Perfluoroalkoxy), PTFE (Polytetrafluoroethylene), FEP (Fluorinated ethylene propylene), or PP (Polypropylene) polymers. It was surprisingly discovered during the development of the current reactor that the material making up the reactor did not have to be a highly corrosive resistant material, as it was discovered that when using even a sanitary grade 316L stainless steel, polished to an internal roughness average (RA) of 15 or less, the PCR reaction was substandard as a result of some type of interference between the PCR reaction mix (buffers, salts, amplicon, polymerase, nucleotides, and primers) and the metallic materials comprising the inside walls of the reaction chambers. In order to optimize the PCR reaction, it is necessary that every surface of reaction chambers 110 coming in contact with the PCR reaction mix be coated with a thin plastic film or be a tube made out of the following materials, such as but not limited to polytetrafluoroethylene (PTFE). PTFE comes in many different types. The types used with success and that are also manufactured with primers such that they are accepted by the FDA for product contact components of drug production equipment, meeting USP Class VI test requirements, are fluorinated-ethylene-propylene, commonly known as FEP and Perfluoroalkoxy, commonly known as PFA. Other plastics could be used depending on whether the material specifications meet requirements for product contact surfaces.

In some embodiments, reaction chambers 110 are positioned in a vertical configuration and the amplicon is loaded from a bottom end toward a top end.

Reactor 100 further includes a variable temperature HT fluid source connected to fluid inlet 104. FIG. 1 shows the variable temperature HT fluid source, such as but not limited to water and glycol, as three fluid baths, a first bath 114 holding an HT fluid at a first temperature, a second fluid bath 116 holding an HT fluid at a second temperature bath and a third fluid bath 118 holding an HT fluid at a third temperature. The HT fluids within the fluid baths may be at temperatures between 2° C. and 99° C. Preferably, the first temperature is a denature temperature between 90-99° C., the second temperature is an anneal temperature between 45-72° C., and the third temperature is an extension temperature between 65-75° C.

Fluid baths 114, 116, 118 are connected to fluid inlet 104 via a first common fluid line 120 and connected to fluid outlet 106 via a second common fluid line 122. First fluid line 120 may also include a booster pump 124 to pump the fluid between 1 and 100 liters/min. through the HT fluid path 108. First fluid bath 114 includes a first outlet valve 126 coupled to inlet 104 via first common fluid line 120 and a first fluid inlet valve 128 coupled to outlet 106 via second common fluid line 122. Second fluid bath 116 includes a second outlet valve 130 coupled to inlet 104 and a second fluid inlet 132 coupled to outlet 106 via second common fluid line 122. Third fluid bath 118 includes a third fluid outlet 134 coupled to inlet 104 first common fluid line 120 and a third fluid inlet 136 coupled to outlet 106 via second common fluid line 122. A controller 138 is connected to fluid outlet valves 126, 130, 134 and fluid inlet valves 128, 132, 136 and controls their opening and closing. By opening and closing fluid outlet valves 126, 130, 134 and fluid inlet valves 128, 132, 136, controller 138 is able to control the fluid temperature in fluid path 108 within reactor housing 102. Controller 138 is capable of controlling temperature ramp rates between 1° C./sec and 10° C./sec. Various thermocouples or other temperature measuring sensors 140 are coupled to the controller 138 to monitor fluid temperatures flowing through reactor 100.

For amplicon amplification, the amplicon is positioned within reaction chambers 110 and sealed. Reaction chambers 110 are positioned within housing 102. First outlet valve 126 and first fluid inlet valve 128 are opened by controller 138 and a HT first fluid 142 from first fluid bath 114 flows through HT fluid path 108 contacting reaction chambers 110. First fluid 142 may be at a denature temperature between 90-99° C. and flows for 5-60 seconds, denaturing the amplicon within chambers 110. After denaturing is complete, controller 138 closes first outlet valve 126 and first fluid inlet valve 128. Controller 138 then opens second outlet valve 130 and second fluid inlet 132 of second fluid bath 116 and a second HT fluid 144 flows through HT fluid path 108 contacting reaction chambers 110. Second HT fluid 144 may be at an anneal temperature between 45-72° C. and flows for 5-45 seconds, annealing the amplicon within chambers 110. After annealing is complete, controller 138 closes second outlet valve 130 and second fluid inlet 132. Controller 138 then opens third outlet valve 134 and third fluid inlet 136 of third fluid bath 118 and a third HT fluid 146 flows through HT fluid path 108 contacting reaction chambers 110. Third HT fluid 146 may be at an extension temperature between 65-75° C. and flows for 90-300 seconds, extending the amplicon within chambers 110. After extension is complete, controller 138 closes third outlet valve 134 and third fluid inlet 136. One amplification cycle of the LEC material within chambers 110 is now complete. If desired, more cycles may be done by repeating the previous steps. In addition, one or more steps may be done separately or repeated. For example, an initial step may be done of flowing the first fluid at a denature temperature for an initial denature step. Another example is a final step may be done of flowing the third HT fluid at an extension temperature for a final extension step. In some embodiments the amplicon may be removed from chambers 110. In other embodiments, the amplicon may remain in chambers 110 and cooled to about 4° C. Once the amplicon has been removed, chambers 110 may be chemically cleaned. While one example of an amplification process cycle has been disclosed above, other process cycles are contemplated using other HT fluid temperatures and times.

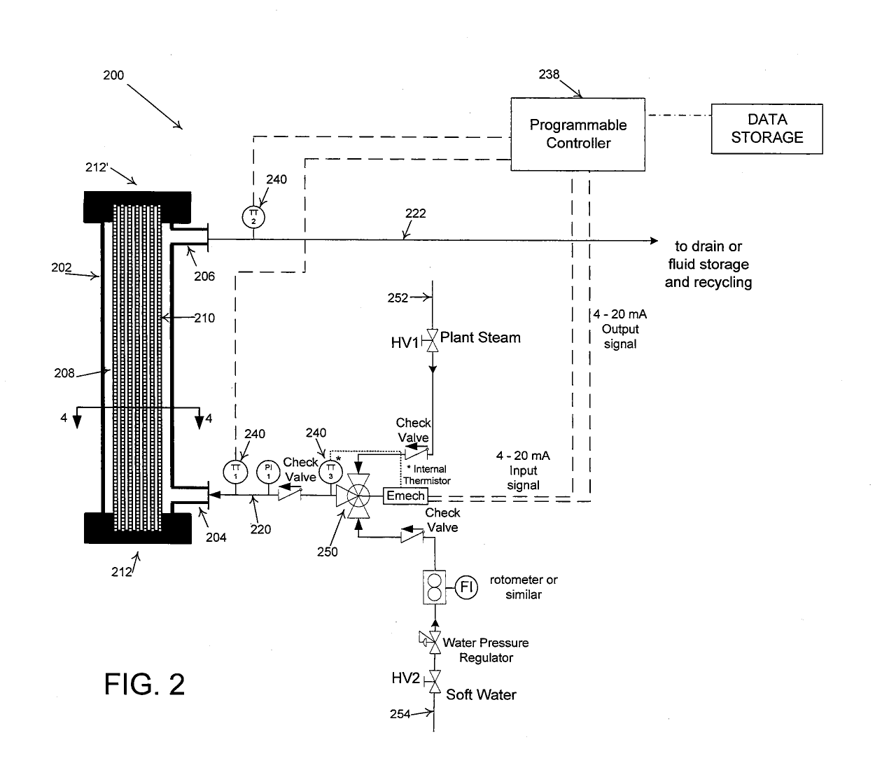

FIG. 2 shows another embodiment of a batch reactor 200 used for amplification of an amplicon. The reactor 200 is similar to reactor 100 except for the variable temperature HT fluid source. While reactor 100 uses fluid baths 114, 116, 118, reactor 200 uses a steam source and a liquid source coupled to a steam/water mixing valve in fluid communication with the fluid inlet, described in more detail below.

Reactor 200 includes a reactor housing 202 having a fluid inlet 204 and a fluid outlet 206 with an HT fluid path 208 there between. A number of reaction chambers 210 are positioned within HT fluid path 208 of housing 202 such that the HT fluid flows around reaction chambers 210. End caps 212 and 212′ may be used to seal ends of housing 202. In some embodiments, end caps 212 and 212′ may also be used to seal reaction chambers 210, if the chambers are not already sealed with the amplicon inside.

Reactor 200 further includes a variable temperature HT fluid source connected to fluid inlet 204. FIG. 2 shows the variable temperature HT fluid source as a steam/water mixing valve 250 coupled to a steam source 252 and a liquid source 254. Steam source 252 may be any suitable steam source, such as plant steam, and liquid source 254 may be any suitable liquid source, such as tap water or soft water. Various shut-off valves, check valves, pressure regulators, flow meters, may be used between steam/water mixing valve 250 and steam source 252 and liquid source 254. Mixing valve 250 is in fluid communication with fluid inlet 204 via a first fluid line 220. Fluid outlet 206 is in fluid communication with second fluid line 222, which may lead to a drain or a fluid storage/recycle system. One example of a suitable steam/water mixing valve is the Emech™ Steam/Water System consisting of a F5 steam water mixer and a G1 electronic actuator, sold by Emech™ Control Limited, Auckland, New Zealand. Mixing valve 250 is capable of mixing steam 252 and liquid 254 to deliver a fluid within first fluid line 220 to the inlet 204 at temperatures between the liquid 254 temperature (with no steam mixed) and 99° C. (mixing steam and liquid). Preferably, mixing valve 250 is configured to provide HT fluid to inlet 204 at temperatures suitable for amplification, including a denature temperature between 90-99° C., an anneal temperature between 45-72° C., and an extension temperature between 65-75° C.

A controller 238 is connected to steam/water mixing valve 250 and controls the opening and closing of an internal valve. By opening and closing the internal valve, controller 238 is able to control the fluid temperature in HT fluid path 208 within reactor housing 202. Controller 238 is capable of controlling temperature ramp rates between 1° C./sec and 10° C./sec. Various thermocouples or other temperature measuring sensors 240 are coupled to the controller 238 to monitor HT fluid temperatures flowing through the reactor 200.

For amplicon amplification, the amplicon is positioned within reaction chambers 210 and sealed. Reaction chambers 210 are positioned within housing 202. Controller 238 controls the valve position of the internal valve in steam/water mixing valve 250 to deliver fluids at suitable temperatures to inlet 204 and HT fluid path 208 for amplification of the amplicon. In a first valve position, steam/water mixing valve 250 delivers a first HT fluid at a denature temperature between 90-99° C. and flows for 5-60 seconds through fluid path 208 contacting reaction chambers 210, denaturing the amplicon within chambers 210. In a second valve position, steam/water mixing valve 250 delivers a second HT fluid at an anneal temperature between 45-72° C. and flows for 15-45 seconds through fluid path 208 contacting reaction chambers 210, annealing the amplicon within chambers 210. In a third valve position, steam/water mixing valve 250 delivers a third HT fluid at an extension temperature between 65-75° C. and flows for 90-300 seconds through fluid path 208 contacting reaction chambers 210, extending the amplicon within chambers 210. The amplification of the amplicon within chambers 210 is now complete. In some embodiments the amplicon may be removed from chambers 210. In other embodiments, the amplicon may remain in chambers 210 and be cooled to about 4° C. Once the amplicon has been removed, chambers 210 may be chemically cleaned. While one example of an amplification process cycle has been disclosed above, other process cycles are contemplated using other fluid temperatures and times.

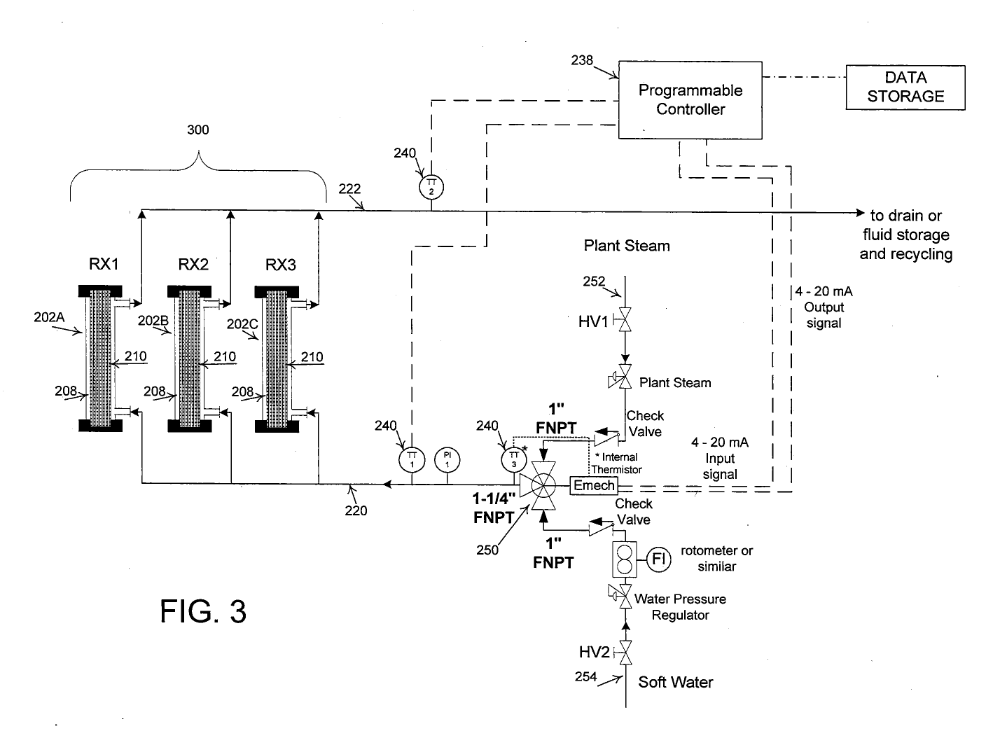

FIG. 3 shows another embodiment of a batch reactor 300 used for amplification of an amplicon. Reactor 300 is similar to reactor 200, except uses multiple reactor housings 202A, 202B, 202C in parallel.

The heat exchangers discussed above and shown in FIG. 1-3 each contemplate the use of a variation of a typical shell and tube heat exchanger. The embodiment illustrated in FIGS. 5-7 are based on the design of a plate heat exchanger. Another type of heat exchanger is the plate heat exchanger. One is composed of multiple, thin, slightly-separated plates that have very large surface areas and fluid flow passages for heat transfer. This stacked-plate arrangement can be more effective, in a given space, than the shell and tube heat exchanger. Advances in gasket and brazing technology have made the plate-type heat exchanger increasingly practical. There are many types of permanently-bonded plate heat exchangers, such as dip-brazed and vacuum-brazed plate varieties, and they are often specified for closed-loop applications such as refrigeration. Plate heat exchangers also differ in the types of plates that are used, and in the configurations of those plates. Some plates may be stamped with “chevron” or other patterns, where others may have machined fins and/or grooves. A variation on the plate heat exchanger is the plate fin heat exchanger. This type of heat exchanger uses “sandwiched” passages containing fins to increase the effectively of the unit. The designs include cross-flow and counter-flow coupled with various fin configurations such as straight fins, offset fins and wavy fins. Plate and fin heat exchangers are usually made of aluminum alloys which provide higher heat transfer efficiency. The material enables the system to operate at a lower temperature and reduce the weight of the equipment. This configuration works well in low temperature operations.

As shown in FIG. 5 heat exchanger 500 comprises a housing or shell 502 having a fluid inlet (not shown) and a fluid outlet (not shown) with a fluid path 508 there between. Heat exchanger 500 further comprises a perforated cage 509 comprising multiple, thin, slightly-separated plates 516 that have very large surface areas and fluid flow passages 518 for heat, shown in FIG. 6. Plates 516 are stacked upon one another and separated by means of spacers 520 so that they are in the range of 0.150+/−0.004 inch apart from each other. Cage 516 maintains and positions reaction chambers 510 in between plates 516. Reaction chambers 510 are constructed from cylindrical tubing that is mechanically compressed to a width that can be positioned between plates 516. The tubing is made out of the following materials, such as but not limited to polytetrafluoroethylene (PTFE). PTFE comes in many different types. The types used with success and that are also manufactured with primers such that they are accepted by the FDA for product contact components of drug production equipment, meeting USP Class VI test requirements, are fluorinated-ethylene-propylene, commonly known as FEP and Perfluoroalkoxy, commonly known as PFA. Other plastics could be used depending on whether the material specifications meet requirements for product contact surfaces.

The inner diameter of the tubing will affect the positioning of the tubes as they are sandwiched between plates 516. Larger diameter tubes will be configured as shown in FIG. 5, whereas smaller diameter tubes may be configured as shown in FIG. 7. Regardless of the configurations, each tube has two ends (not shown) and when these ends are sealed a reaction chamber 510 or 710 (FIGS. 5 and 7, respectively) is formed. In one example, each tube end may be sealed thus allowing the PCR mixture containing the desired amplicon to be loaded into the tubes prior to positioning the tube within cage 509. In this instance, tube ends could then be sealed after the PCR reaction mixture is loaded into the tube but prior to the tube being positioned with cage 509. Alternatively, the tube could be positioned with the cage 509 and then tube end could be sealed by end cap (not shown). In another embodiment, a tube is positioned within cage 509 and the end of the tube is effectively sealed by virtue of being in contact with the end cap. The PCR reaction mixture is then loaded into the tube through the open tube end and then reaction chamber 510 or 710 (FIGS. 5 and 7, respectively) is formed by sealing the open tube end by positioning an end cap over the top of housing 502. Reaction chambers 510 or 710 (FIGS. 5 and 7, respectively) within reactor housing 502 are thus positioned within fluid path 108 of housing 502 such that the HT fluid flows around reaction chambers 510 or 710 (FIGS. 5 and 7, respectively) thereby transferring heat from the HT fluid to the PCR reaction mixture confined within reaction chambers 510 or 710 (FIGS. 5 and 7, respectively) through the reaction chamber side walls.

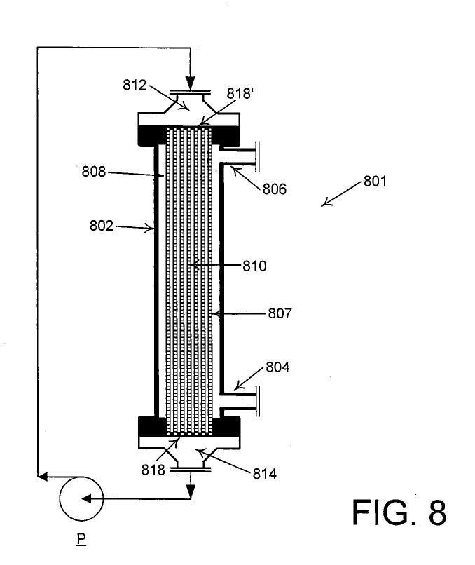

FIG. 8 illustrates a heat exchanger 800 that may be substituted with any of the heat exchangers discussed previously when used with a batch reactor embodiment described above. Heat exchanger 800 differs from the heat exchange reactors described previously in that there are not multiple reaction chambers. Instead, heat exchange unit 801 comprises a tube bundle 807 positioned within reactor housing or shell 802 having an HT fluid inlet 804 and an HT fluid outlet 806 with a fluid path 808 there between. Tube bundle 807 comprises a plurality of tubes wherein the ends of each tube are connected to plenums (an inlet plenum 812 and an outlet plenum 814) through holes in tube sheets 818 and 818′ all of which creates a reaction chamber 810. The PCR reaction mix is introduced into reaction chamber 810 by way of inlet plenum 812 travels through tube bundle 807 where heat is transferred causing the reaction to take place. The PCR reaction mix then departs tube bundle 807 through outlet plenum 814 and is circulated by way of a pump P back to inlet plenum 812. The PCR reaction mix will continue to be circulated while the HT fluid is being passed through the heat exchanger 800 as discussed previously. As discussed previously it is necessary to coat any part of the reaction chamber 810 coming in contact with the PCR reaction mix with a thin plastic film out of the following materials, such as but not limited to polytetrafluoroethylene (PTFE). PTFE comes in many different types. The types used with success and that are also manufactured with primers such that they are accepted by the FDA for product contact components of drug production equipment, meeting USP Class VI test requirements, are fluorinated-ethylene-propylene, commonly known as FEP and Perfluoroalkoxy, commonly known as PFA. Other plastics could be used depending on whether the material specifications meet requirements for product contact surfaces. Alternatively, suitable heat exchangers made entirely of PFA may be purchased from sources including; Entegris, Billerica, Mass., Ametek, Paoli, Pa., and Heateflex, Arcadia, Calif., thus eliminating the need to coat the surfaces of a heat exchanger made from metallic materials such as, but not limited to sanitary grade 316L stainless steel.

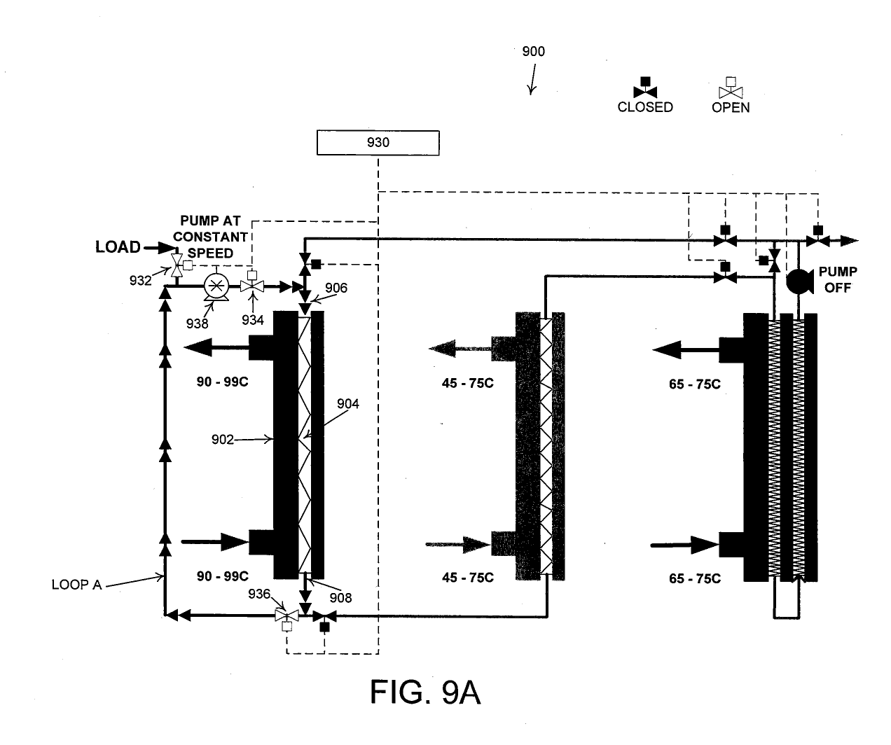

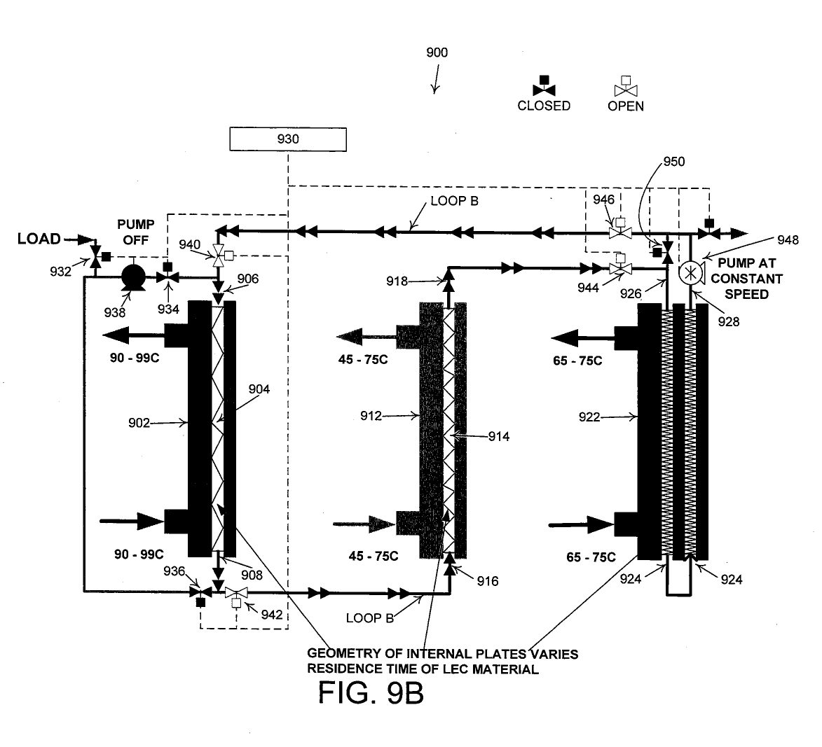

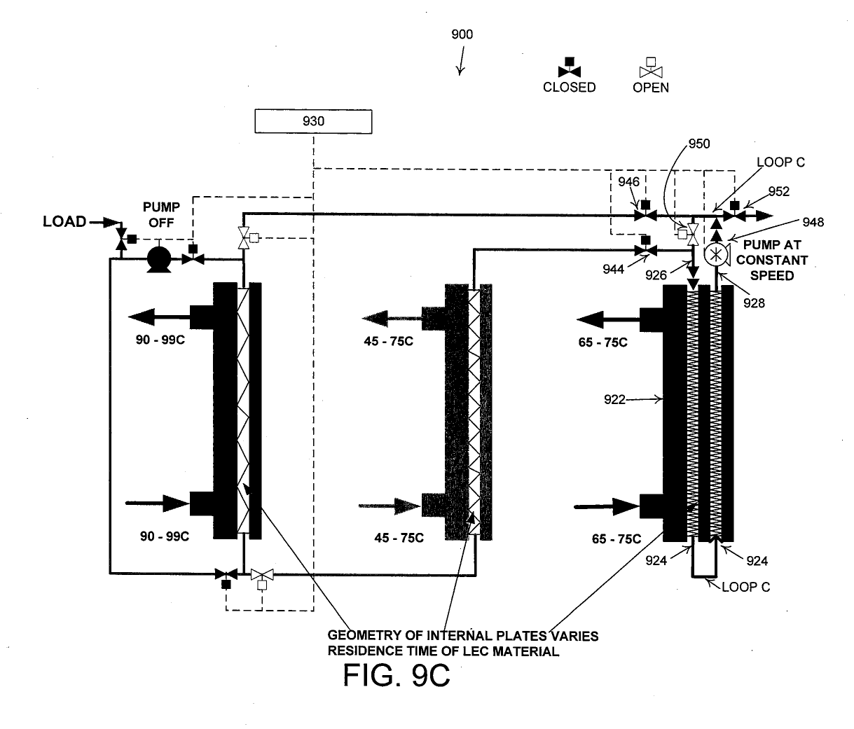

FIGS. 9A,94B and 9C show one embodiment of a continuous flow reactor 400 used for amplification of a LEC material passing through heat exchanger modules at different temperatures, heating and cooling the amplicon for amplification. Reactor 900 includes first, second and third heat exchanger modules 902, 912, 922. First heat exchanger module 902 is constructed of a first plate heat exchanger having a PCR reaction mixture containing the amplicon fluid path 904 passing through the first heat exchanger module 902 from a first fluid inlet 906 to a first fluid outlet 908. Second heat exchanger module 912 is constructed of a second plate heat exchanger having an amplicon fluid path 914 passing through second heat exchanger module 912 from a second fluid inlet 916 to a second fluid outlet 918. Third heat exchanger module 922 is constructed of a third plate heat exchanger having an amplicon fluid path 924 passing through third heat exchanger module 922 from a third fluid inlet 926 to a third fluid outlet 928. The plate heat exchangers may be either electrically heated/cooled or may use a fluid passing through the plate heat exchanger for heating or cooling. Suitable plate heat exchangers may be purchased from sources including ITT Standard, Cheektowaga, N.Y.; Mueller, Springfield, Mo.; Alfa Laval Corporation AB, Sweden; GEA PHE Systems York, Pa.; and APV Getzville, N.Y.

In the embodiment shown in FIGS. 9A, 9B and 9C, the plate heat exchanger modules use a fluid for heating/cooling. First heat exchanger module 902 is uses a first HT fluid at a denature temperature between 90-99° C., second heat exchanger module 912 uses a second HT fluid at an anneal temperature between 45-72° C. and third heat exchanger module 922 uses a third HT fluid at an extension temperature between 65-75° C. One or more pumps are used to pump the PCR reaction mixture containing the amplicon through the different heat exchanger modules 902, 912, 922 of the reactor. The plate heat exchanger plates can be individually shaped as to effect the residence time through each section (longer or shorter) and the PCR reaction mixture containing the amplicon will continuously flow through numerous plates at a constant flow rate.

Various valves are opened and closed to direct the PCR reaction mixture containing the amplicon to the fluid paths 904, 914, 924 of the different heat exchanger modules 902, 912, 922, depending on the amplification cycle. A controller 930 may be coupled to various valves and to control the opening and closing of the valve. Controller 930 may also control one or more pumps in the reactor.

One embodiment of an amplification cycle is shown in FIGS. 9A, 9B and 9C in which reactor 900 has three distinct “loops” used for amplifying the amplicon. FIG. 9A shows “Loop A” in which the amplicon is circulated only through amplicon fluid path 904 of heat exchanger module 902 for an initial denature step. FIG. 9B shows “Loop B” in which the amplicon is circulated through amplicon fluid paths 904, 914, 924 of all three heat exchanger modules 902, 912, 922 for denature, anneal and extension steps. FIG. 9C shows “Loop C” in which the amplicon is circulated only through amplicon fluid path 924 of heat exchanger module 922 for a final extension step.

For amplicon amplification, PCR reaction mixture containing the amplicon is introduced into Loop A through a valve 932 then closed. Valves 934, 936 are opened and the PCR reaction mixture containing the amplicon is pumped around Loop A through fluid path 904 of first heat exchanger module 902 using a pump 938 for an initial denature step at a denature temperature between 90-99° C., as shown in FIG. 9A. Pump 938 may pump the PCR reaction mixture containing the amplicon at a constant speed through Loop A. After the initial denature step, valves 934 and 936 of Loop A are closed and valves 940, 942, 944, 946 of Loop B are opened. The PCR reaction mixture containing the amplicon is pumped around Loop B through fluid path 904 of heat exchanger module 902 at a denature temperature between 90-99° C. for 5-60 seconds, through fluid path 914 of second heat exchanger module 912 at an anneal temperature between 45-72° C. for 15-45 seconds, and fluid path 924 of heat exchanger module 922 at an extension temperature between 65-75° C. for 90-300 s using a pump 948, as shown in FIG. 9B. Once around Loop B would be one cycle. Any number of cycles may be done, for example, between 1 and 100. Once the amplification is complete, valves 944 and 946 of Loop B are closed and valve 950 Loop C is opened and the PCR reaction mixture containing the amplicon flow through fluid path 924 of heat exchanger module 922 for a final extension step. Once the final extension step is complete, valve 952 is opened and the amplified material is harvested. In other embodiments, the initial denature step and/or the final extension step may be lengthen or eliminated.

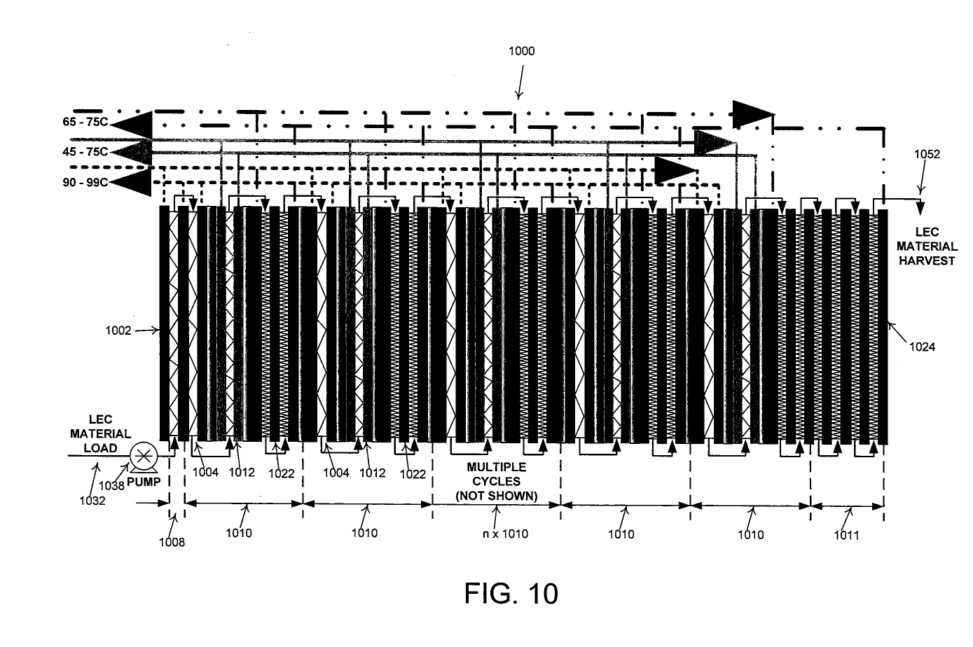

FIG. 10 show another embodiment of a continuous flow reactor 500 used for amplification of an amplicon passing through heat exchanger modules at different temperatures, heating and cooling the PCR reaction mixture containing the amplicon for amplification. Reactor 1000 is a true continuous flow reactor in that PCR reaction mixture containing the amplicon enters an inlet, flows in a continuous fluid path through multiple heat exchanger modules where is the amplicon is amplified and exits an outlet as amplified material. In the embodiment shown, separately configured heat exchanger modules may be used in reactor 1000 to effect residence time within each individual module. Reactor 1000 includes one first heat exchanger module 1002 for an initial denature step 1008, multiple sets of first, second and third heat exchanger modules 1004, 1012 and 1022 arranged in series, and a third heat exchanger module 1024 for a final extension step 1011. While FIG. 10 shows five cycles 1010 through sets of first, second and third heat exchanger modules 1004, 1012 and 1022, any number of cycles can be done for amplification by adding or subtracting sets of heat exchanger modules 1004, 1012 and 1022.

In use, the PCR reaction mixture containing the amplicon is loaded in an inlet 1032 and goes through heat exchanger module 1002 for an initial denature 1008. The PCR reaction mixture containing the amplicon then goes through multiple cycles 1010 by going through multiple sets of first, second and third heat exchanger modules 1004, 1012 and 1022 arranged in series. After a final extension step 1011 through third heat exchanger modules 1024, the amplified amplicon is harvested as it exits through the outlet 1052. Pump 1038 pumps the material through the continuous fluid path. The number of cycles 1010 is determined by the amplification and may be between 2 and 100.

While the exemplary embodiments have been described in some detail, by way of example and for clarity of understanding, those of skill in the art will recognize that a variety of modification, adaptations, and changes may be employed. Hence, the scope of the present invention should be limited solely by the appending claims.

What is claimed is:

1. A batch reactor for amplification of an amplicon, the reactor comprising:
a housing having a fluid path with a fluid inlet and a fluid outlet;
one or more reaction chambers, having an inside and outside surface, suitable for housing the PCR reaction mixture containing the amplicon the reaction chambers being positioned within the housing in thermal communication with a heat transfer fluid flowing in the fluid path to heat and/or cool the reaction chambers wherein the inside surface of the reaction chambers is a plastic material;
a variable temperature fluid source in fluid communication with the fluid inlet, wherein the fluid source is configured to provide fluid at temperatures suitable for amplification, including a denature temperature, an anneal temperature, and an extension temperature; and
a controller coupled to the variable temperature fluid source, the controller being configured to control a fluid temperature within the fluid path by controlling the variable temperature fluid source.

2. The reactor of claim 1, wherein the variable temperature fluid source comprises a plurality of fluid baths at different temperatures with outlet valves in fluid communication with the fluid inlet of the housing, the controller being configured to open and close the outlet valves of the fluid baths to control the fluid temperature within the housing.

3. The reactor of claim 2, wherein the plurality of fluid baths further include inlet valves in fluid communication with the fluid outlet of the housing, the controller being configured to open and close the inlet valves to allow fluid to return to the fluid baths after traveling through the housing.

4. The reactor of claim 1, wherein the variable temperature fluid source comprises a steam source and a liquid source coupled to a steam/water mixing valve in fluid communication with the fluid inlet, the controller being configured to adjust the steam/water mixing valve to control the fluid temperature within the housing.

5. The reactor of claim 1, wherein the variable temperature fluid source is configured to deliver fluid at temperatures between 2° C. and 99° C.

6. The reactor of claim 1, wherein the denature temperature is between 90-99° C., the anneal temperature is between 45-72° C., and the extension temperature between 65-75° C.

7. The reactor of claim 1, wherein the controller is capable of controlling temperature ramp rates between 1° C./sec and 10° C./sec.

8. The reactor of claim 1, wherein the reaction chambers are sealable.

9. The reactor of claim 1, wherein the reaction chambers are constructed of a PFA material.

10. The reactor of claim 1, wherein the reaction chambers are cylindrical in shape having a diameter between 0.05 and 0.5 inches any length up to 60 inches.

11. The reactor of claim 1, wherein the reaction chambers have a volume between 0.001 and 1 liter.

12. The reactor of claim 1, further comprising a fluid pump configured to pump the fluid between 1 and 100 liters/min. through the fluid path.

13. The reactor of claim 1, wherein the reaction chambers are a vertical configuration and are loaded from a bottom end toward a top end.

14. The reactor of claim 1, further comprising one or more thermocouples or other temperature measuring sensors coupled to the controller configured to monitor fluid temperatures.

15. A method of amplification of an amplicon using a batch reactor, the method comprising:
providing a batch reactor comprising:
a housing having a fluid path with a fluid inlet and a fluid outlet;
one or more reaction chambers suitable for housing the amplicon, the reaction chambers being positioned within the housing in thermal communication with a fluid flowing in the fluid path to heat and/or cool the reaction chambers;
a variable temperature fluid source in fluid communication with the fluid inlet, wherein the fluid source is configured to provide fluid at temperatures suitable for amplification, including a denature temperature, an anneal temperature, and an extension temperature; and
a controller coupled to the fluid source, the controller being configured to control a fluid temperature within the fluid path by controlling the variable temperature fluid source;
inserting the material within the one or more reaction chambers; and
heating and cooling the material within the reaction chambers for an amplification process cycle by flowing fluid in the fluid path at a plurality of temperatures and time periods from the variable temperature fluid source.

16. The method of claim 15, wherein the amplification process cycle includes at least a denature temperature between 90-99° C. for 5-60 seconds, an anneal temperature between 45-72° C. for 15-45 seconds, and an extension temperature between 65-75° C. for 90-300 seconds.

17. The method of claim 15, further comprising removing the amplicon from the reaction chambers when the amplification process cycle is complete.

18. The method of claim 17, further comprising chemically cleaning the reaction chambers.

19. The method of claim 15, further comprising cooling the reaction chambers to about 4° C. after the amplification process cycle is complete.

20. The method of claim 15, wherein inserting the LEC within the one or more reaction chambers includes preloading the LEC within the reaction chambers and placing in storage prior to amplification.

21. A continuous flow reactor for amplification of an amplicon, the reactor comprising:
a plurality of heat exchanger modules constructed of plate heat exchangers, each heat exchanger module having an amplicon fluid path combinable into a continuous amplicon fluid path passing through the plurality of heat exchanger modules;
a plurality of fluid sources configured to provide heating/cooling fluids to a heating/cooling fluid path of each heat exchanger module, wherein each of the heat exchanger modules is heated/cooled to temperatures suitable for amplification, including a denature temperature, an anneal temperature, and an extension temperature; and
a controller coupled to the plurality of fluid sources, the controller being configured to control a temperature within each of the plurality of heat exchanger modules by controlling a fluid temperature and/or fluid flow rate through the heating/cooling fluid path.

22. The reactor of claim 21, wherein the amplicon completes one process cycle after flowing in the continuous amplicon fluid path through the plurality of heat exchanger modules.

23. The reactor of claim 22, wherein the heating/cooling fluid path within each heat exchanger module is configured to heat or cool the amplicon for a portion of the process cycle.

24. The reactor of claim 21, wherein the plurality of fluid sources comprise a plurality of fluid baths at different temperatures.

25. The reactor of claim 21, wherein each of the fluid sources comprise a steam source and a liquid source coupled to a steam/water mixing valve in fluid communication with the heating/cooling fluid path, the controller being configured to adjust the steam/water mixing valve to control the fluid temperature.

26. The reactor of claim 21, wherein a fluid flow rate of the amplicon in the amplicon fluid path of each heat exchanger module is dependent upon the temperature of the fluid flowing through the heating/cooling fluid path of the heat exchanger module.

27. The reactor of claim 21, wherein a fluid flow rate of the amplicon in the amplicon fluid path through each of the heat exchanger modules is the same.

28. The reactor of claim 21, wherein a fluid residence time of the amplicon in the amplicon fluid path through each of the heat exchanger modules is different.

29. The reactor of claim 21, wherein the plurality of fluid sources are configured to heat fluids to temperatures between 2° C. and 99° C.

30. The reactor of claim 21, wherein the denature temperature is between 90-99° C., the anneal temperature is between 45-72° C., and the extension temperature between 65-75° C.

31. A continuous flow reactor for amplification of an amplicon, the reactor comprising:
a plurality of heat exchanger modules, each heat exchanger module having an amplicon fluid path combinable into a continuous amplicon fluid path passing through the plurality of heat exchanger modules from a fluid inlet and a fluid outlet; and
a plurality of fluid sources configured to provide heating/cooling fluids to a heating/cooling fluid path of each heat exchanger module at temperatures suitable for amplification, including a denature temperature, an anneal temperature, and an extension temperature.

32. The reactor of claim 31, wherein the heat exchanger modules are constructed of plate heat exchangers.

33. The reactor of claim 31, wherein the plurality of heat exchanger modules are in sets of three, a first heat exchanger module at a denature temperature, a second heat exchange module at an anneal temperature and a third heat exchanger module at an extension temperature.

34. The reactor of claim 31, wherein the denature temperature is between 90-99° C., the anneal temperature is between 45-72° C., and the extension temperature between 65-75° C.

35. A method of amplification of an amplicon using a continuous flow reactor, the method comprising:
providing a continuous flow reactor comprising:
a plurality of heat exchanger modules constructed of plate heat exchangers, each heat exchanger module having an amplicon fluid path combinable into a continuous amplicon fluid path passing through the plurality of heat exchanger modules; and
a plurality of fluid sources configured to provide heating/cooling fluids to a heating/cooling fluid path of each heat exchanger module, wherein each of the heat exchanger modules is heated/cooled to temperatures suitable for amplification, including a denature temperature, an anneal temperature, and an extension temperature;
flowing the amplicon through the plurality of heat exchanger modules within the continuous amplicon fluid path; and
heating or cooling the amplicon in the amplicon fluid path within each heat exchanger module, wherein one amplification process cycle is complete after the amplicon is heated to a denature temperature, an anneal temperature, and an extension temperature.

36. The method of claim 35, wherein the plurality of heat exchanger modules includes a first heat exchanger module at a denature temperature, a second heat exchange module at an anneal temperature and a third heat exchanger module at an extension temperature.

37. The method of claim 35, wherein one amplification process cycle includes at least a denature temperature between 90-99° C. for 5-60 seconds, an anneal temperature between 45-72° C. for 15-45 seconds, and an extension temperature between 65-75° C. for 90-300 seconds.

38. The method of claim 35, wherein the continuous flow reactor further comprises a controller coupled to the plurality of fluid sources, the controller being configured to control a temperature within each of the plurality of heat exchanger modules by controlling a fluid temperature and/or fluid flow rate through the heating/cooling fluid path.

39. The method of claim 35, wherein the amplicon is subjected to more than one process cycle.

40. The reactor of claim 1, wherein the amplicon is DNA.

41. The reactor of claim 1, wherein the amplicon is RNA.

42. The reactor of claim 1, wherein the DNA is a sequence that may be expressed.

43. The reactor of claim 1, wherein the sequence when expressed is an antigen from a virus, or bacteria.

44. The reactor of claim 1, wherein the amplified amplicon may be used as a vaccine.

45. The reactor of claim 1, wherein the amplified amplicon is an adjuvant, vaccine, or an inhibitory or stimulatory DNA.

46. The reactor of claim 1, wherein the plastic material is PTFE, PFA, or FEP.
